(19) United States
(12) Patent Application Publication
(10) Pub. No.: US 2009/0157078 A1
(43) Pub. Date: Jun. 18, 2009

(54) Apparatus and Methods of Repairing Bone Defects

(76) Inventor: Edward J. Mikol, Myrtle Beach, SC (US)

Correspondence Address: THE H.T. THAN LAW GROUP, WATERFRONT CENTER SUITE 560, 1010 WISCONSIN AVENUE NW, WASHINGTON, DC, 20007, US

(21) Appl. No.: 12/336,150

(22) Filed: Dec. 16, 2008

Related U.S. Application Data
(60) Provisional application No. 61/014,133, filed on Dec. 17, 2007.

(51) Int. Cl. A61B 17/58

(52) U.S. Cl. 606/62; 606/64

(57) ABSTRACT

An apparatus and method for repairing osseous defects in such bones as the humerus, femur, ulna and long bones. A first embodiment presents one or more cannulated screws affixed to an intramedullary nail, which is installed in a humerus having an intraosseous void. In a second embodiment, one or more cannulated screws alone are inserted into a proximal humerus having an intraosseous void. Other embodiments teach one or more cannulated screws inserted into a damaged femur and attached to a plate or medullary rod. Another embodiment presents a number of cannulated screws used to attach a plate to a fractured long bone. In a final embodiment, a cannulated screw is installed in damaged ulna. In all embodiments, a bioresorbable or non-bioresorbable cement is injected into a hollow channel of the cannulated screw and extruded into the bone to stabilize the weakened bone and secure the position of a fixation device.

### FIELD OF THE INVENTION

This invention relates to an apparatus and method for repairing bone defects or augmenting the fixation of a device into or onto bone. In particular, the invention relates to using cannulated screws, either alone or in conjunction with a nail, plate, rod or other fixation device, to treat osseous voids or weaknesses or to secure fixation devices that may otherwise fail due to weak purchase in bone.

### BACKGROUND OF THE INVENTION

Osseous voids, which threaten the structural integrity of a bone, are caused by trauma, infection, congenital malformation, tumor growth, or degenerative diseases. As discussed in U.S. Pat. No. 7,045,125, bone grafts are conventionally used to regenerate and heal the affected bone. Alternatively, as disclosed in the '125 patent, affected bones can be treated by filling the osseous voids with biologically active composites that are injected into the osseous voids.

U.S. Pat. No. 5,776,194, which is incorporated herein by reference in its entirety, also discloses the use of bone cement or so-called “void filler” to treat osseous voids. In particular, the '194 patent discloses that bone cement can be used to fill intraosseous voids associated with intraosseous proximal humerus fractures.

However, none of the related art discloses the use of cannulated screws to introduce a filling type cement or glue.

### SUMMARY OF THE INVENTION

This invention relates to an apparatus and method for treating conditions that threaten the structural integrity of bone, e.g., osseous voids and fractures caused by trauma or structural weaknesses resulting from congenital defects, infection or disease.

In a first embodiment, one or more cannulated screws are affixed to a bone nail, such as an intramedullary nail, which is installed in a proximal humerus with an intraosseous void. In a second embodiment, one or more cannulated screws alone are installed in a proximal humerus with an intraosseous void. In both the first and second embodiments, a bioresorbable glue or cement, or non-bioresorbable cement such as polymethyl methacrylate, is injected into a hollow longitudinal channel of the one or more cannulated screws. Subsequently, the bioresorbable or non-bioresorbable glue or cement extrudes into the intraosseous void, interdigitates with porous bone, or fills a naturally occurring cavity.

A third embodiment of the invention presents one or more cannulated screws inserted into a femur through a plate affixed to the outside of the bone. A bioresorbable or non-bioresorbable cement or glue is deposited into a longitudinal channel of one or more of the screws and is extruded into the bone. In accordance with this embodiment, a cavity or void may or may not exist in the bone; the cement or glue either interdigitates with the pores of the bone to secure the fixation of the screw and hence the fixation of the plate or fills an intraosseous void or cavity to fortify the weakened bone.

In a fourth embodiment of the present invention, one or more cannulated screws are inserted through a plate into a long bone to secure the fixation of the plate to the bone. In accordance with this embodiment, the screws pass through the naturally occurring medullary cavity of the bone. A bioresorbable or non-bioresorbable glue or cement is injected into the longitudinal channel of one or more of the screws and extrudes into the medullary cavity, securing the fixation of the plate to the bone and fortifying the structural integrity of the bone.

In a fifth embodiment of the present invention, a cannulated screw is affixed to a rod inserted into the medullary cavity of a femur. The cannulated screw passes into the head of the femur to secure a fracture occurring at the femoral neck. A bioresorbable or non-bioresorbable glue or cement is injected into the longitudinal channel of the cannulated screw and is extruded into the bone. As in the third embodiment, a void or cavity may or may not exist in the bone. If no intraosseous void or cavity exists, the glue or cement interdigitates with the pores of the bone to strengthen the fixation of the screw and rod; if an intraosseous void or cavity does exist, the glue or cement fills the void or cavity to repair the defect and secure the position of the screw.

A sixth embodiment of the present invention teaches a cannulated screw inserted into the proximal extremity of the ulna and passing into the medullary cavity to secure a fracture at the olecranon. A bioresorbable or non-bioresorbable glue or cement is injected into the longitudinal channel of the cannulated screw and is subsequently extruded into the bone, interdigitating with the porous bone at the proximal end of the ulna and also partially filling the medullary cavity of the ulna.

In a seventh embodiment of the present invention, cannulated screws pass through a long bone having a facture, e.g. a spiral facture. To secure the facture, the screws are inserted at angled directions to cross the path of the fracture and pass entirely through the bone and out of the side of the shaft opposite their entry points. A bioresorbable or non-bioresorbable glue may be introduced into the medullary cavity via exit slots located along the length of the cannulated screws.

### BRIEF DESCRIPTION OF THE DRAWINGS

In the accompanying drawings, which form a part of the specification and are to be read in conjunction therewith and in which like reference numerals are used to indicate like parts in the various views:

FIG. 1 depicts a first embodiment of the invention applied to a proximal humeral cavity, incorporating one or more cannulated screws attached to an intramedullary nail;

FIG. 2 shows a second embodiment of the invention applied to a proximal humeral cavity, incorporating one or more cannulated screws;

FIGS. 3A and 3B depict details of a cannulated screw and optional washer;

FIG. 4 shows a third embodiment of the invention in which at least one cannulated screw is inserted into a femur to strengthen the weakened bone and to secure the attachment of a plate;

FIG. 5 shows a fourth embodiment of the invention applied to a long bone, incorporating a number of cannulated screws affixing a plate to said long bone;

FIG. 6 depicts a fifth embodiment of the invention in which a cannulated screw is attached to an intramedullary rod and inserted into the head of a femur weakened by a fracture at the femoral neck;

FIG. 7 shows a sixth embodiment of the present invention applied to an ulna having a fracture at the olecranon process, incorporating a cannulated screw passing through the proximal extremity and into the medullary cavity of the ulna; and

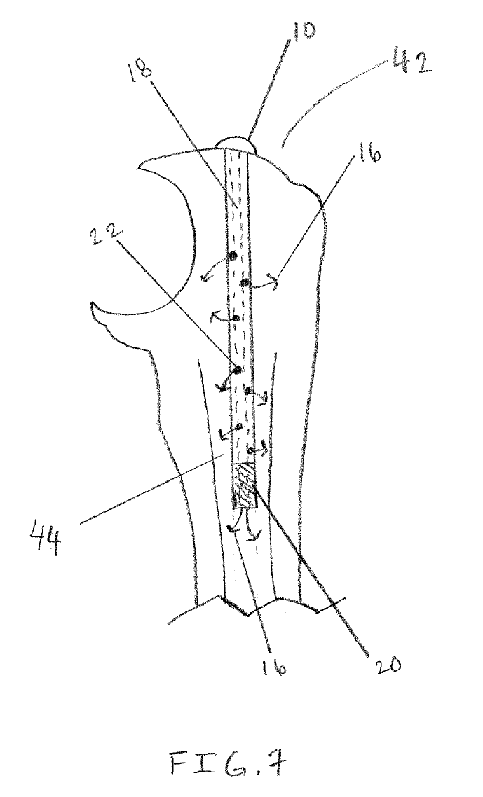

FIG. 8 shows an application of the seventh embodiment, in which a spiral fracture of a long bone is secured by two cannulated screws, each passing entirely through the bone in angled directions across the path of the facture.

### DETAILED DESCRIPTION OF THE INVENTION

The structure and method of the present invention are described in the context of treating fractures and intraosseous voids or fractures in bones such as the humerus, femur, ulna and long bones. It will be understood by one of ordinary skill in the art that such voids weaken the structural integrity of the bone, possibly resulting in pathologic or impending fractures. However, the present invention is not limited to treating fractures of the humerus, femur, ulna and long bones, but may be used for treating osseous voids of other bones, either pathologic or naturally occurring (e.g., the medullary canal). Moreover, in addition to treating intraosseous voids, the present invention may also be used to augment the fixation of plates, screws, rods and other devices, to fortify weak bone that requires stabilization and to treat nonunions, malunions, bone tumors, and cavitary lesions.

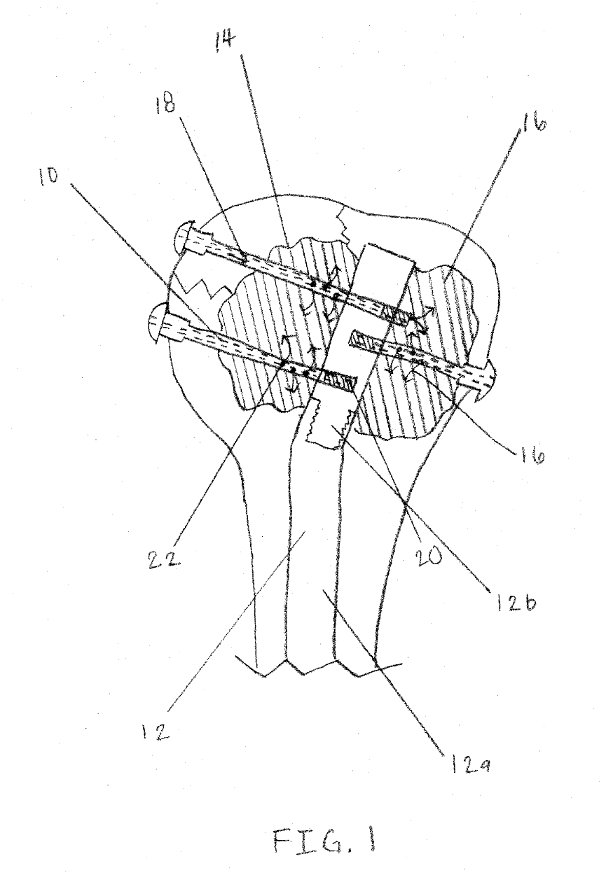

FIG. 1. illustrates the first embodiment of the present invention. After an intramedullary nail 12 is installed in the proximal humerus with intraosseous void 14, one or more cannulated screws 10 are affixed to intramedullary nail 12. The intramedullary nail 12, as illustrated in FIG. 1, is an intramedullary rod having a stem member 12a and an extension member 12b. The cannulated screws 10 can be attached to extension member 12b or stem 12a. The threaded distal ends 20 of cannulated screws 10 can terminate in stem member 12a or extension member 12b, or they may pass through stem member 12a or extension member 12b to terminate inside the bone or osseous void. Further discussion of the intramedullary rod, including how stem member 12a and extension member 12b are connected, is found in U.S. Pat. No. 5,776,194, which was previously incorporated by reference in its entirety, and patents cited therein. However, it should be noted that the present invention is not limited simply to use in conjunction with an intramedullary rod as depicted in FIG. 1, but can be used with any suitable intramedullary device known in the art.

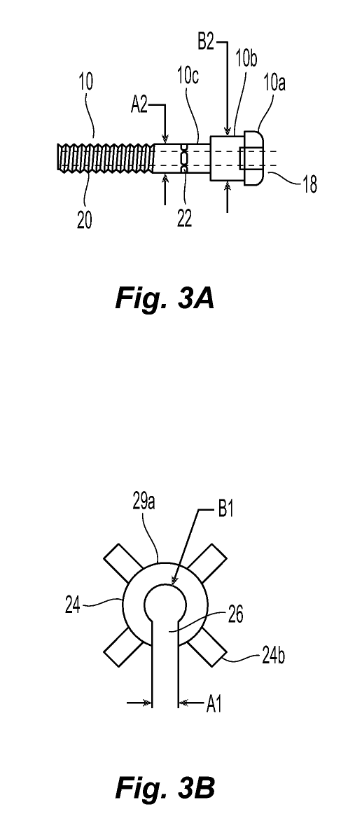

The cannulated screw 10, as best shown in FIG. 3A, includes a hollow longitudinal channel 18 for injection, passage, and extrusion of a bioresorbable or non-bioresorbable glue or cement 16. The cannulated screw 10 may further include a hollow threaded end 20 for attaching to intramedullary nail 12 or another piece of bone, and exit slots 22 for the extrusion of bioresorbable or non-bioresorbable glue or cement 16. The glue or cement can also be extruded from hollow end 20. The cannulated screw 10 can be constructed from metal, plastic, composite, a bioresorbable material (such as polyglycolic acid), or other suitable material.

A bioresorbable or non-bioresorbable glue or cement 16, suitable for bonding and/or structural support, can be introduced into the proximal end of hollow longitudinal channel 18. The glue or cement 16 can comprise polymethylmethacrylate, calcium phosphate, calcium sulfate, or other freely injectable substance that hardens in the body. After the glue or cement 16 is injected into the proximal end of cannulated screw 10 via longitudinal channel 18, it is extruded into intraosseous void 14 through the hollow distal end 20, if cannulated screw 10 includes an opening at its distal end, of hollow longitudinal channel 18 or exit slots 22. Subsequent to curing, the bioresorbable or non-bioresorbable glue or cement 16 can securely reconstruct the proximal humerus defect. Suitable bioresorbable materials include, but are not limited to, Boneplast™ Bone Void Filler from Interpore Cross International, Inc., Biomet™ Calcigen™ from Biomet Orthopedics, Inc., HydroSet™ from Stryker, Inc., and B One™ XR Bone Void Filler from Arthrotek.

In accordance with the present invention, the one or more cannulated screws 10 as discussed in each embodiment of the present invention, can be used in conjunction with washer-like structure 24, shown in greater detail in FIG. 3B, to distribute the force of attachment over a wider area of bone and surrounding soft tissue than could be distributed by a screw alone. As shown in FIG. 3B, the claw washer 24 includes a slot 26 having a width A1 slightly larger than the diameter A2 of the shaft 10c of cannulated screw 10. The inner diameter B1 of the washer 24 is slightly greater than B2 to provide clearance for the shaft 10b of the cannulated screw 10, but smaller than the diameter of head 10a. The respective dimensions of slot 26, A1, A2, B1 and B2 permit the claw washer 24 to be inserted laterally under the screw head 10a after the cannulated screw 10 is started into bone. Further, since A1 is less than B2, the tightening of cannulated screw 10 causes B1 to engage B2, locking the washer 24 into place and preventing the washer from slipping out. The washer 24, for this example, has a plurality of relatively shallow prongs 24b extending outward, which grip soft tissue as well as bone. It will be understood by one skilled in the art that the prong pattern of claw washer 24 could be circular, or irregular, or rectangular. Further, prongs 24b may be perpendicular to the portion 24a of claw washer 24 as shown, or may extend from 24a at an angle (not shown). The ends of prongs 24b are preferably pointed, but this is not required to implement this invention. Prongs 24b may have a limited bending ability, thereby allowing one or more to be adjusted or removed as needed, depending on the fracture pattern. Alternatively, cannulated screw 10 can be used in conjunction with any suitable washer such as a standard washer having a closed hole.

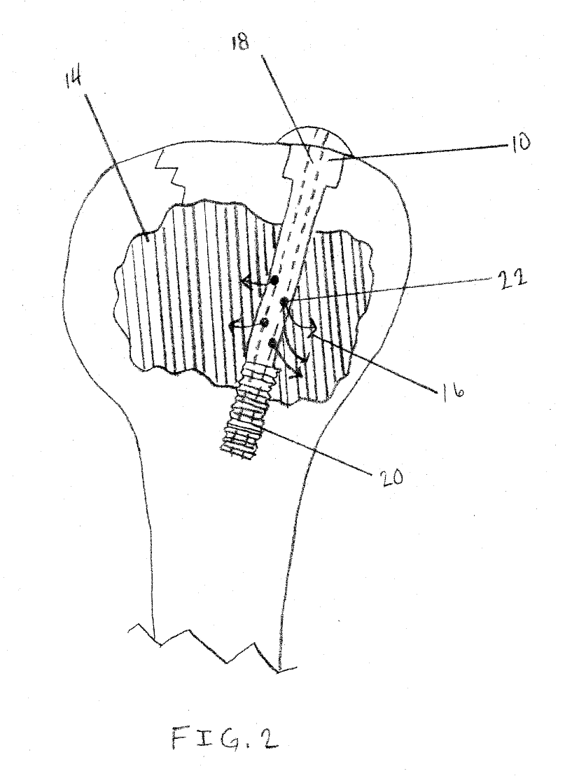

A second embodiment for treating a broken bone, such as a proximal humerus with intraosseous void 14, as illustrated in FIG. 2, uses one or more cannulated screws 10. More specifically, the one or more cannulated screws 10 contain exit slots 22 in addition to hollow longitudinal channel 18 and hollow threaded end 20. After one or more cannulated screws 10 are installed in the bone, a bioresorbable or non-bioresorbable glue or cement 16 can be injected into the proximal end of hollow longitudinal channel 18. Subsequently, the glue or cement 16 is extruded into intraosseous void 14 from hollow distal end 20 of hollow longitudinal channel 18 or exit slots 22. After curing, the one or more cannulated screws 10 are then incorporated into the cement construct, adding stability to the reconstructed humerus.

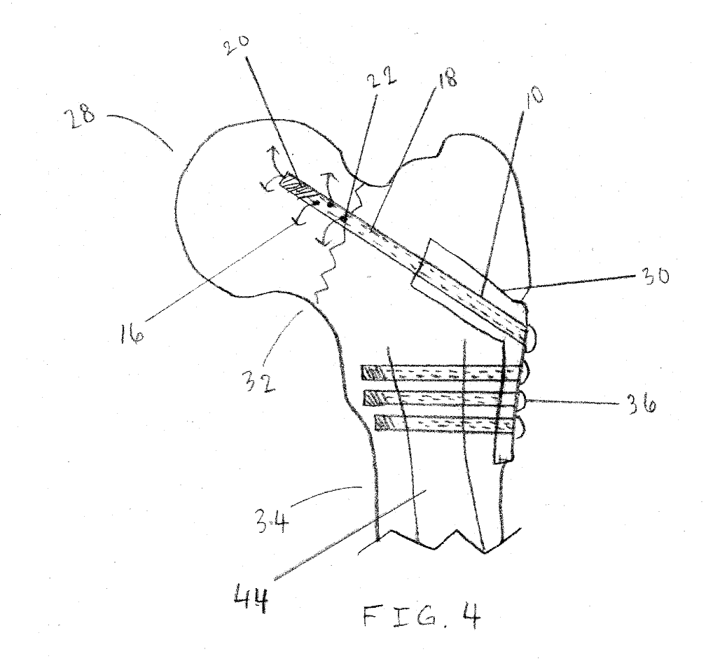

In a third embodiment of the present invention, illustrated in FIG. 4, one or more cannulated screws 10 are inserted into a femur through a plate 30 affixed to the outside of the bone to secure a fracture at the femoral neck 32. At least one cannulated screw 10 slides through plate 30 and passes into femoral head 28. A bioresorbable or non-bioresorbable glue or cement 16 is deposited into the proximal end of cannulated screw 10 and through hollow longitudinal channel 18. If no osseous void or cavity exists, glue or cement 16 is extruded through exit slots 22 or hollow distal end 20 of hollow longitudinal channel 18 and subsequently interdigitates with the porous bone of femoral neck 32 or femoral head 28. If an osseous void or cavity does exist at the proximal end of the femur, glue or cement 16 is extruded through exit slots 22 or hollow distal end 20 of hollow longitudinal channel 18 and fills said osseous void or cavity. Upon curing, glue or cement 16 helps to strengthen the fixation of cannulated screw 10 and plate 30, further stabilizing the bone. In accordance with this embodiment, one or more screws 36 may be used to affix plate 30 to body 34 of the femur. These screws pass through medullary cavity 44 of the femur and may or may not be cannulated.

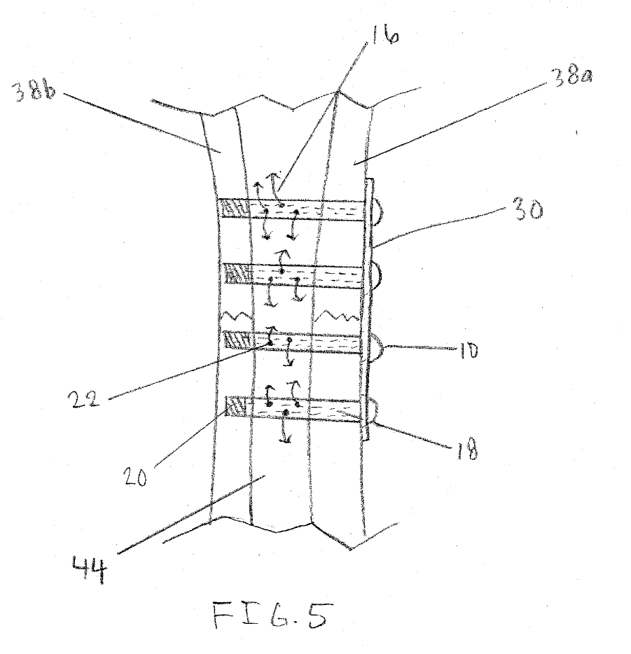

A fourth embodiment of the present invention teaches a number of cannulated screws 10 used to join a plate 30 to the outside of a long bone in order to secure a fracture of the bone, as shown in FIG. 5. In accordance with this embodiment, cannulated screws 10 are inserted into plate 30, pass through cortex 38a, pass through medullary cavity 44 and are secured by means of distal threaded ends 20 of cannulated screws 10 into cortex 38b. A bioresorbable or non-bioresorbable glue or cement 16 is injected into hollow longitudinal channel 18 and is extruded through exit slots 22 into medullary cavity 44 of the long bone. Cured glue or cement 16 fortifies the bone and helps to secure the fixation of cannulated screws 10 and plate 30.

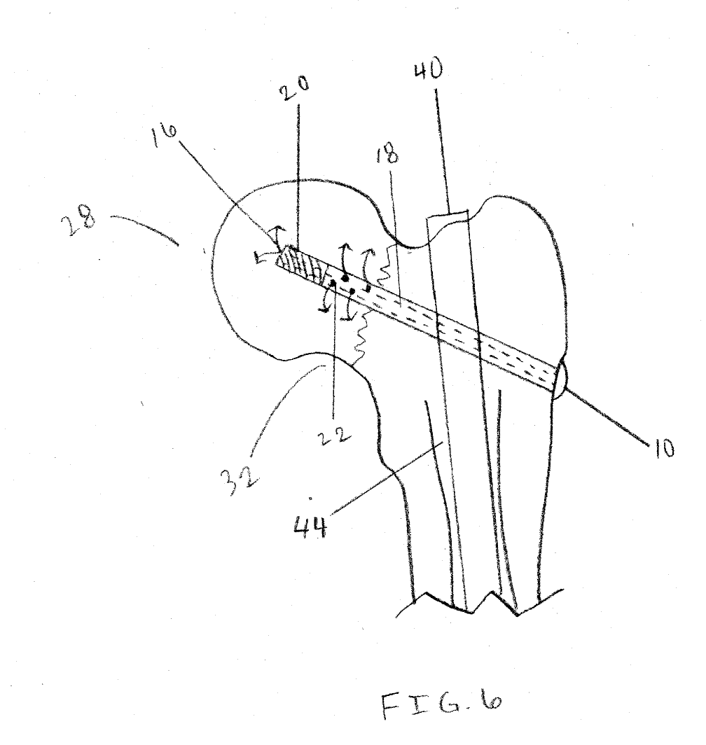

In yet another embodiment of the present invention, illustrated in FIG. 6, cannulated screw 10 is inserted through rod 40 installed in medullary cavity 44 of a femur and into femoral head 28 to secure a fracture at the femoral neck. The proximal end of the femur, including femoral neck 32 and femoral head 28, may or may not include an intraosseous void or cavity. Bioresorbable or non-bioresorbable glue or cement 16 is deposited into cannulated screw 10 through hollow longitudinal channel 18. Glue or cement 16 extrudes from cannulated screw 10 through exit slots 22 or hollow distal end 20 of hollow longitudinal channel 18. Depending on the existence of an osseous void or cavity, glue or cement 16 may fill an intraosseous void or cavity occurring at femoral neck 32 or femoral head 28 or may interdigitate with the porous bone of the proximal end of the femur. Subsequently, cured glue or cement 16 helps to hold cannulated screw 10 firmly in place and hence fortifies the bone that has been weakened by a fracture at the femoral neck.

In a sixth embodiment of the present invention, a cannulated screw 10 is installed through the proximal extremity 42 of an ulna having a fracture at the olecranon process. Cannulated screw 10 is inserted through proximal extremity 42 and into medullary cavity 44 of the ulna. Bioresorbable or non-bioresorbable glue or cement 16 is deposited into the proximal end of cannulated screw 10, through hollow longitudinal channel 18 and extruded out of exit slots 22 and hollow distal end 20 of hollow longitudinal channel 18. Exit slots 22 occur along the length of cannulated screw 10 so that glue or cement 16, when extruded, meets both the porous bone of proximal extremity 42 and medullary cavity 44 that begins at a location distal to proximal extremity 42. Glue or cement 16 subsequently interdigitates with the porous bone of proximal extremity 42 and also fills at least part of medullary cavity 44 of the ulna, fortifying the weak bone and incorporating cannulated screw 10 into the bone and cement construct.

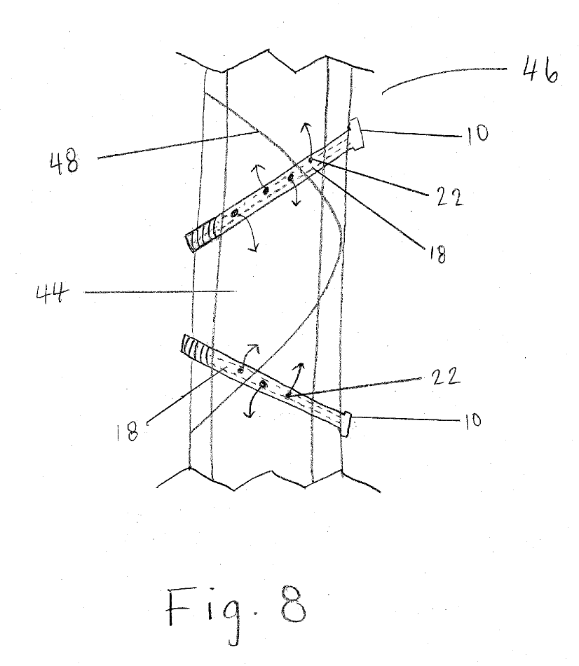

FIG. 8 depicts an application of a seventh embodiment of the present invention. A facture 48 of long bone 46, in this case a spiral fracture, is secured by a number of cannulated screws 10. Each screw 10 is inserted into the shaft of long bone 46, passes through the path of the fracture, and exits through the side of the shaft opposite the side of the entry point. A portion of the distal end of cannulated screw 10 exits long bone 46. In this embodiment the distal end is not hollow, but is closed. A bioresorbable or non-bioresorbable glue may be injected into the cannulated screw and extruded through exit slots 22. The glue or cement then fills medullary cavity 48, or any porous bone or intraosseous void present, to harden and further secure the fractured bone. Because the distal end of cannulated screw 10 is closed, glue is prevented from exiting the distal end and combining with other tissues.

While it is apparent that the illustrative embodiments of the invention disclosed herein fulfill the objectives of the present invention, it is appreciated that numerous modifications and other embodiments may be devised by those skilled in the art. Additionally, feature(s) and/or element(s) from any embodiment may be used singly or in combination with feature(s) and/or element(s) from other embodiment(s). For example, the intramedullary rod apparatus in FIG. 1 could be unitary, that is, stem member 1 and extension member 2 could include a one-piece structure formed of metal, plastic or a bioresorbable material. Therefore, it will be understood that the appended claims are intended to cover all such modifications and embodiments, which would come within the spirit and scope of the present invention.

1. A bone repairing device comprising:
a) a bone fixation device and
b) one or more cannulated screws attached to the bone fixation device wherein said one or more cannulated screws comprise a hollow longitudinal channel, one or more exit slots, and an optional hollow threaded end.

2. A bone repairing device according to claim 1 further comprising a washer disposed between the bone nail, plate, or rod and each of the one or more cannulated screws.

3. A bone repairing device according to claim 1 wherein the one or more cannulated screws are constructed from a bioresorbable material.

4. A bone repairing device according to claim 1 wherein the bone fixation device is an intramedullary nail.

5. A bone repairing device according to claim 4 wherein the intramedullary nail has a stem member and an extension member.

6. A bone repairing device according to claim 1 wherein the bone fixation device is an intramedullary rod.

7. A method for restoring an osseous void comprising:
a) providing a bone repairing device according to claim 1; and
b) injecting a bone cement into said one or more cannulated screws.

8. A method for restoring an osseous void according to claim 7 wherein the one or more cannulated screws are constructed from a bioresorbable material.

9. A method for restoring an osseous void according to claim 7 wherein said bone cement comprises polymethylmethacrylate, calcium phosphate, or calcium sulfate.

10. A method for augmenting a bone fixation device comprising:
a) providing a bone repairing device according to claim 1; and
b) injecting a bone cement into said one or more cannulated screws.

11. A method for augmenting a bone fixation device according to claim 10 wherein the one or more cannulated screws are constructed from a bioresorbable material.

12. A method for augmenting a bone fixation device according to claim 10 wherein said bone cement comprises polymethylmethacrylate, calcium phosphate, or calcium sulfate.

13. A method for restoring an osseous void comprising:
a) inserting, through the osseous void, one or more cannulated screws comprising a hollow longitudinal channel, one or more exit slots, and an optional hollow threaded end; and
b) injecting a bone cement into said one or more cannulated screws.

14. A method for restoring an osseous void according to claim 13 wherein the one or more cannulated screws are constructed from a bioresorbable material.

15. A method for restoring an osseous void according to claim 13 wherein said bone cement comprises polymethylmethacrylate, calcium phosphate, or calcium sulfate.

16. A bone repairing device comprising one or more cannulated screws attached to a bone, wherein said one or more cannulated screws comprise a hollow longitudinal channel, one or more exit slots, and an optional hollow threaded end, and wherein said cannulated screw is sized and dimensioned to allow bone cement to be injected into the bone.

17. The bone repairing device of claim 16, wherein the one or more cannulated screws are constructed from a bioresorbable material.
